(19) United States
(12) Patent Application Publication
(10) Pub. No.: US 2024/0065595 A1
(43) Pub. Date: Feb. 29, 2024

(54) METHOD AND SYSTEM FOR CALCULATING EMOTIONAL INDICATORS BASED ON PUPIL-WAVE

(71) Applicant: Beijing University Of Technology, Beijing (CN)

(72) Inventors: Mi LI, Beijing (CN); Bin HU, Beijing (CN); Shengfu LV, Beijing (CN); Jiaming KANG, Beijing (CN); Wei ZHANG, Beijing (CN)

(73) Assignee: Beijing University Of Technology, Beijing (CN)

(21) Appl. No.: 18/387,062

(22) Filed: Nov. 6, 2023

Related U.S. Application Data
(63) Continuation of application No. PCT/CN2021/133488, filed on Nov. 26, 2021.

(30) Foreign Application Priority Data
Oct. 11, 2021 (CN) 202111178895.4

(51) Int. Cl. A61B 5/16; A61B 5/00

(52) U.S. Cl. CPC A61B 5/163; A61B 5/7264; A61B 5/7278

(57) ABSTRACT

A method and system for calculating emotional indicators based on pupil-wave are provided. The operation of calculating emotional indicators based on a pupil-wave may include the following operations. A pupil-wave is collected when a subject is in a calm state and set as a standard pupil-wave; Obtaining emotional indicators and corresponding emotions used to measure a mental state. The pupil-wave of the subject in each emotional state is collected and set as an emotional pupil-wave; According to the standard pupil-wave, a bandwidth and differential pupil-wave corresponding to each emotion are calculated; The standard, emotional, bandwidth and differential pupil-wave are input into a pre-trained deep convolutional neural network to obtain index values of emotional indicators.

### CROSS REFERENCE TO THE RELATED APPLICATIONS

This application is a continuation application of International Application No. PCT/CN2021/133488, filed on Nov. 26, 2021, which is based upon and claims priority to Chinese Patent Application No. 202111178895.4, filed on Oct. 11, 2021, the entire contents of which are incorporated herein by reference.

### TECHNICAL FIELD

The present invention relates to the field of computer technology, in particular to a method and system for calculating emotional indicators based on pupil-wave.

### BACKGROUND

Considering the progression of the economy and society, individuals are displaying increasingly elevated expectations concerning the quality and efficacy of their educational pursuits, professional endeavors, and daily existence. These growing demands have led to a heightened sensitivity towards the environments in which people engage in these activities, consequently inducing deviations in emotional indicators due to the resultant discomfort experienced within these contexts. If a long-term abnormal emotional state is not alleviated, it may lead to psychological abnormalities, and eventually develop into anxiety disorders and depression. Emotional abnormalities usually present as emotional disorders: main emotional symptoms of stress include feeling psychologically tense, experiencing palpitations, restlessness and emotional instability; main emotional symptoms of anxiety include feeling fear, disturbed, nervous and apprehensive; main emotional symptoms of depression include feeling low mood, distress, anhedonia, and decreased interest. If the severity of abnormal emotional indicators cannot be promptly and accurately detected and assessed, and if timely psychological intervention cannot be implemented, there is a high likelihood that it may develop into anxiety disorder or depression, damaging physical health.

Physical health examination is a necessary means to ensure early detection and treatment of diseases. However, the current physical health examination is mainly based on physiological equipment for blood glucose, ECG, blood lipids and other physiological health checks. There is a lack of examination and evaluation equipment for emotional indicators.

Currently, self-rating scales are the primary method used to assess mood indicators. For example, a PSTR self-rating scale is used to assess stress, a PHQ-9 self-rating scale is used to assess depression, and a GAD-7 self-rating scale is used to assess anxiety. Using these scales requires a certain level of cultural knowledge and understanding. As the self-scales consist of multiple items, each item includes more than three options, making an assessment method for emotional indicators subjective. At the same time, the GAD-7 self-rating anxiety scale, PHQ-9 self-rating depression scale, and other self-rating scales lack direct emotional indicators directly related to mood, the evaluation and discrimination are not directly related to emotional experiences, resulting in the relatively poor assessment accuracy.

### SUMMARY

A purpose of present invention is to provide a method and system for calculating emotional indicators based on a pupil-wave; the method and system can accurately calculate the emotional indicators and assess an emotional state of a subject. Then it can accurately distinguish a mental state, and timely intervention to ensure the mental health and physical health of the subject.

To achieve the above purpose, present invention adopts the following technical solution:

A method for calculating emotional indicators based on a pupil-wave, including the following steps:
S1: A pupil-wave is collected when a subject is in a calm state and set as a standard pupil-wave;
S2: Obtaining emotional indicators and corresponding emotions used to measure the mental state;
S3: The pupil-wave of the subject in each emotional state are collected and set as an emotional pupil-wave;
S4: According to the standard pupil-wave, bandwidth and differential pupil-wave corresponding to each emotion are calculated;
S5: The standard, emotional, bandwidth and differential pupil-wave are input into a pre-trained deep convolutional neural network to obtain index values of emotional indicators;
Among them, the pupil-wave represent curve of pupil diameter or pupil area.

Preferably, Step S2, the emotional indicators include one or more of depression indicators, anxiety indicators and stress indicators:
The depression indicators correspond to multiple emotions, including happiness and sadness;
The anxiety indicators correspond to multiple emotions, including happiness and threat;
The stress indicators correspond to multiple emotions, including happiness and tension.

Preferably, in Step S3, collecting the pupil-wave, specifically comprising the steps:
S31: A curve of the subject's pupil diameter or pupil area is obtained to acquire a raw pupil-wave;
S32: Points of the pupil≤50 pixels are removed, and the raw pupil-wave including missing values;
S33: An adjacent mean difference method is used to fill in the missing values and then the pupil-wave is obtained. A calculation formula for the adjacent mean difference method is as follows:

P(t)=(P(t−1)+P(t+1))/2 (Formula 1)

P(t) is the value of the pupil-wave at the t moment.

Preferably, calculation formulas of the bandwidth pupil-wave are as follows:

$$\tilde{P} = \frac{1}{m} {\sum }_{t = 1 ∼ m} {p}_{0} ( t ) \qquad ( Formula 2 )$$

$${EP}_{i} ( t ) = {P}_{i} ( t ) - \tilde{P}$$

P₀(t) is a value of the standard pupil-wave at a t second, m is an acquisition time, \tilde{P} is a mean value, Pᵢ(t) is a value of the pupil-wave corresponding to an i emotion at the t second, EPᵢ(t) is a value of the bandwidth pupil-wave corresponding to the i emotion at the t second.

Preferably, A calculation formula for the differential pupil-wave is as follows:

DPᵢ(t)=Pᵢ(t+1)−Pᵢ(t) (Formula 3)

Pᵢ(t) is the value of the emotional pupil-wave corresponding to the i emotion at the t second, Pᵢ(t+1) is the value of the t+1 second, DPᵢ(t) is the value of the differential pupil-wave corresponding to the i emotion at the t second.

Preferably, the pre-trained deep convolutional neural network specifically comprises:
Pupil-wave channel: it is used to receive the standard pupil-wave and emotional pupil-wave and extract features through a convolutional neural network;
Bandwidth pupil-wave channel; it is used to receive the bandwidth pupil-wave and extract features through a convolutional neural network;
Differential pupil-wave channel: it is used to receive differential pupil-wave and extract features through a convolutional neural network;
Fully connected layer: it is used to receive features extracted from the pupil-wave channel, bandwidth pupil-wave channel, and differential pupil-wave channel, and outputs index values of emotional indicators.

Present invention also provides a system for calculating emotional indicators based on pupil-wave, the system comprising:
The first collection module; it is used to collect the pupil-wave when the subject is in a calm state and set as the standard pupil-wave;
The first obtaining module: it is used to measure the mental state emotional indicators;
The second obtaining module: it is used to obtain multiple emotions corresponding to emotional indicators;
The second collection module: it is used to collect the pupil-wave of the subject in each emotional state and set as an emotional pupil-wave;
The data processing module: It is used to calculate the index values of emotional indicators based on the standard pupil-wave and emotional pupil-wave.

Therefore, present invention provides a method and system for calculating emotional indicators based on pupil-wave using the above-described structure, resulting in beneficial effects as follows:
Present invention can obtain the dynamic change of pupils by collecting the pupil-wave of the subject. It can objectively measure the subject's ability to experience calm, happiness, sadness, threat, and tension. Compared with existing self-rating scales and taking photos to obtain the state pictures of the subject, this invention reduces the influence of subjective factors. It can accurately calculate the emotional indicators of the subject, assess their emotional states, thereby accurately distinguish their mental states, and intervene promptly to ensure the mental and physical health of the subject.

### BRIEF DESCRIPTION OF THE DRAWINGS

FIG. 1 illustrates a flow chart of a method used in present invention to calculate emotional indicators based on a pupil-wave.

FIG. 2 illustrates a schematic diagram of a system structure used in present invention for calculating emotional indicators based on a pupil-wave.

### DETAILED DESCRIPTION OF THE EMBODIMENTS

The further explanations of a technical solution of present invention through accompanying figures and examples of implementation are as follows:

Unless otherwise defined, technical or scientific terms used in the invention shall be understood in the usual sense by persons of average skill in the field to which the invention belongs. The terms “first,” “second,” and similar words used in the present invention do not indicate any specific order, quantity, or importance but are merely used to distinguish between different components. Words such as “including” or “containing” mean that the component or object mentioned before the word includes the component or object listed after the word and its equivalent, and does not exclude other components or objects. Words such as “including” or “containing” and similar expressions imply that the components or objects mentioned before the word encompass the components or objects listed after the word and their equivalents, and does not exclude other components or objects. The terms “setup,” “installation,” and “connection” should be interpreted broadly, such as fixed connections, detachable connection or integrated connection. It can be a mechanical connection or an electrical connection, and can be either direct or indirect through an intermediate medium. Additionally, the terms may refer to connections between two components. Words like “up,” “down,” “left,” “right,” and so on are simply used to express relative position relationships. The relative position relationship may change accordingly when the absolute position of the described object changes.

### Examples of Implementation

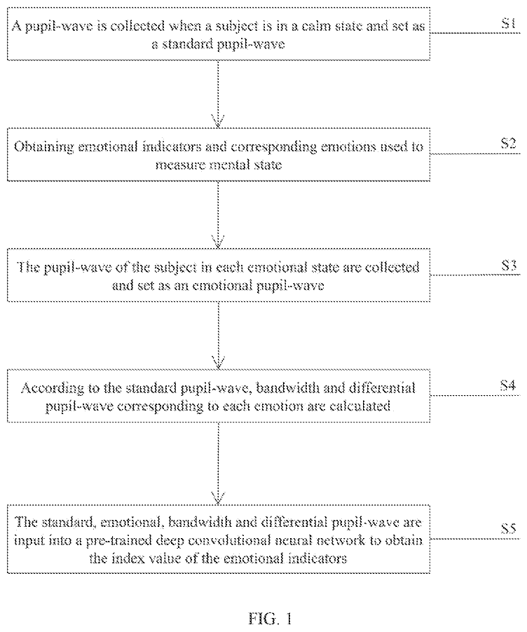

As shown in FIG. 1, present invention provides a method for calculating emotional indicators based on a pupil-wave. This process involves following steps:
1. A pupil-wave is collected when a subject is in a calm state and set as a standard pupil-wave;
2. Obtaining emotional indicators and corresponding emotions used to measure a mental state;
The emotional indicators include one or more of depression, anxiety and stress indicators.

The depression indicators correspond to multiple emotions, including happiness and sadness;
The anxiety indicators correspond to multiple emotions, including happiness and threat;
The stress indicators correspond to multiple emotions, including happiness and tension.

By using emotional indicators as an objective standard for mental state assessment, measuring the pupil-wave associated with different emotions in the subject fills the previous deficiency in emotional assessment of mental state.

3. The pupil-wave of the subject in each emotional state are collected and set as an emotional pupil-wave;

The method of generating pupil-wave includes:
(1) Generate virtual reality scenes corresponding to calm, sadness, happiness, threat, and tension, respectively;
(2) The virtual reality scenes are edited. Scenes that are too short cannot effect on the emotions of the subject, and too long will interfere with the subject's ability to experience emotional changes. When the length of the scene is 1 minute and 30 seconds, the test purpose can be achieved;
(3) The edited virtual reality scenes are provided to the subject according to the set order, and the scenes are played according to the order of calm, sadness, happiness, threat, and tension, which can minimize the mutual influence of different kinds of scenes.
(4) A pupil curve of the subject is collected over time.

Collecting pupil-wave, specifically comprising the steps:
S31: A curve of the subject's pupil diameter or pupil area over time is obtained to acquire a raw pupil-wave;
S32: Points of the pupil≤50 pixels are removed, and the raw pupil-wave including missing values;
S33: An adjacent mean difference method is used to fill in the missing values and then the pupil-wave is obtained. A calculation formula for the adjacent mean difference method is as follows:

P(t)=(P(t−1)+P(t+1))/2 (Formula 1)

P(t) is the value of the pupil-wave at the t moment. The values one second before and one second after the missing value are averaged as the missing value.

4. According to the standard pupil-wave, bandwidth and differential pupil-wave corresponding to each emotion are calculated;

Using an average of standard pupil-wave as the standard, the bandwidth pupil-wave corresponding to each emotional pupil-wave is calculated. Formulas for calculating the bandwidth pupil-wave is as follows:

$$\tilde{P} = \frac{1}{m} {\sum }_{t = 1 ∼ m} {p}_{0} ( t ) \qquad ( Formula 2 )$$

$${EP}_{i} ( t ) = {P}_{i} ( t ) - \tilde{P}$$

P₀(t) is the value of the standard pupil-wave at the t second, m is the acquisition time of, \tilde{P} is the mean value, Pᵢ(t) is the value of the pupil-wave corresponding to the i emotion at the t second, EPᵢ(t) is the value of the bandwidth pupil-wave corresponding to the i emotion at the t second.

A calculation formula for the differential pupil-wave is as follows:

DPᵢ(t)=Pᵢ(t+1)−Pᵢ(t) (Formula 3)

Pᵢ(t) is the value of the emotional pupil-wave corresponding to the i emotion at the t second, Pᵢ(t+1) is the value of the t+1 second, DPᵢ(t) is the value of the differential pupil-wave corresponding to the i emotion at the t second.

5. The standard, emotional, bandwidth and differential pupil-wave are input into a pre-trained deep convolutional neural network to obtain index values of emotional indicators.

The pre-trained deep convolutional neural network, specifically comprises:
Pupil-wave channel: it is used to receive the standard pupil-wave and emotional pupil-wave and extract features through a convolutional neural network;
Bandwidth pupil-wave channel: it is used to receive the bandwidth pupil-wave and extract features through a convolutional neural network;
Differential pupil-wave channel: it is used to receive differential pupil-wave and extract features through a convolutional neural network;
Fully connected layer: it is used to receive features extracted from the pupil-wave channel, bandwidth pupil-wave channel, and differential pupil-wave channel, and outputs index values of emotional indicators.

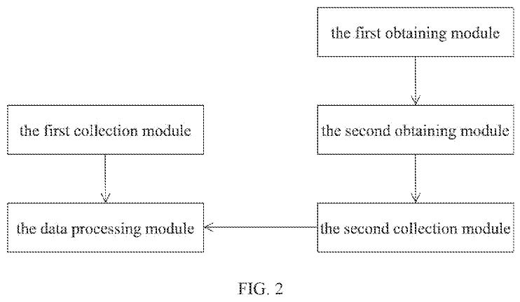

As shown in FIG. 2, present invention also provides a system for calculating emotional indicators based on pupil-wave, the system comprising:
The first collection module: it is used to collect a pupil-wave when a subject is in a calm state and set as standard pupil-wave;
The first obtaining module: it is used to measure mental state emotional indicators;
The second obtaining module: it is used to obtain multiple emotions corresponding to emotional indicators;
The second collection module: it is used to collect the pupil-wave of the subject in each emotional state and set as an emotional pupil-wave;
The data processing module: it is used to calculate index values of emotional indicators based on the standard pupil-wave and emotional pupil-wave.

Among them, the pupil-wave represent curves of pupil diameter or pupil area over time.

Therefore, present invention provides a method and system for calculating emotional indicators based on pupil-wave, using above-mentioned structure. Present invention can obtain the dynamic change of pupils by collecting pupil-wave of the subject. It can objectively measure the subject's ability to experience calm, happiness, sadness, threat, and tension. Compared with existing self-rating scales and taking photos to obtain the state pictures of the subject, this invention reduces the influence of subjective factors. It can accurately calculate the emotional indicators of the subject, assess their emotional states, thereby accurately distinguish their mental states, and intervene promptly to ensure the mental and physical health of the subject.

Finally, it should be noted that the above embodiments are provided solely for the purpose of illustrating the technical scheme of the present invention and should not be considered as limiting its scope. Although reference has been made to the preferred embodiments to provide detailed explanations of the present invention, those with ordinary technical knowledge in the field should comprehend that they are still free to modify or replace the technical scheme of the invention. However, such modifications or substitutions should not cause the modified technical scheme to deviate from the spirit and scope of the technical scheme of the present invention.

What is claimed is:

1. A method for calculating emotional indicators based on a pupil-wave, comprising the following steps:
collecting a pupil-wave while a subject is in a calm state and establishing the pupil-wave as a standard pupil-wave;
acquiring emotional indicators and emotions corresponding to the emotional indicators configured to measure a mental state;
gathering pupil-wave data from the subject experiencing various emotional states and designating the pupil-wave data as an emotional pupil-wave;
calculating a bandwidth pupil-wave and a differential pupil-wave, wherein the bandwidth pupil-wave and the differential pupil-wave are linked to each of the emotions based on the standard pupil-wave;
inputting standard, emotional, bandwidth, and differential pupil-wave data into a pre-trained deep convolutional neural network to derive index values of the emotional indicators;
wherein the pupil-wave represent temporal curves of a pupil diameter or pupil area.

2. The method for calculating the emotional indicators based on the pupil-wave according to claim 1, wherein the emotional indicators comprise at least one of depression indicators, anxiety indicators and stress indicators:
the depression indicators correspond to a plurality of first emotions, comprising happiness and sadness;
the anxiety indicators correspond to a plurality of second emotions, comprising happiness and threat;
the stress indicators correspond to a plurality of third emotions, comprising happiness and tension.

3. The method for calculating the emotional indicators based on the pupil-wave according to claim 2, wherein the steps for collecting pupil-wave comprise:
deriving a raw pupil-wave by capturing temporal changes in the subject's pupil diameter or pupil area;
removing points where a pupil is ≤50 pixels, resulting in the raw pupil-wave with missing values;
employing an adjacent mean difference method to fill in the missing values, and the pupil-wave is obtained: a calculation formula for the adjacent mean difference method is as follows:
P(t)=(P(t−1)+P(t+1))/2 (Formula 1)
P(t) is a value of the pupil-wave at the t moment.

4. The method for calculating the emotional indicators based on the pupil-wave according to claim 3, wherein calculation formulas of the bandwidth pupil-wave are as follows:
$$\tilde{P} = \frac{1}{m} {\sum }_{t = 1 ∼ m} {p}_{0} ( t ) \qquad ( Formula 2 )$$
$${EP}_{i} ( t ) = {P}_{i} ( t ) - \tilde{P}$$
P₀(t) is a value of the standard pupil-wave at a t second, m is an acquisition time, \tilde{P} is a mean value, Pᵢ(t) is a value of the pupil-wave corresponding to an i emotion at the t second, EPᵢ(t) is a value of the bandwidth pupil-wave corresponding to the i emotion at the t second.

5. The method for calculating the emotional indicators based on the pupil-wave according to claim 4, comprising a calculation formula for the differential pupil-wave, the calculation formula is as follows:
DPᵢ(t)=Pᵢ(t+1)−Pᵢ(t) (Formula 3)
Pᵢ(t) is a value of the emotional pupil-wave corresponding to the i emotion at the t second, Pᵢ(t+1) is a value of the emotional pupil-wave corresponding to the i emotion at the t+1 second, DPᵢ(t) is a value of the differential pupil-wave corresponding to the i emotion at the t second.

6. The method for calculating the emotional indicators based on the pupil-wave according to claim 5, wherein the pre-trained deep convolutional neural network comprises:
a pupil-wave channel, wherein the pupil-wave channel is configured to receive the standard pupil-wave and the emotional pupil-wave and extract first features through a first convolutional neural network;
a bandwidth pupil-wave channel, wherein the bandwidth pupil-wave channel is configured to receive the bandwidth pupil-wave and extract second features through a second convolutional neural network;
a differential pupil-wave channel, wherein the differential pupil-wave channel is configured to receive the differential pupil-wave and extract third features through a third convolutional neural network;
a fully connected layer, wherein the fully connected layer is configured to receive the first, second, and third features extracted from the pupil-wave channel, the bandwidth pupil-wave channel, and the differential pupil-wave channel, and outputs the index values of the emotional indicators.

7. A system for calculating emotional indicators based on pupil-wave, configured for the method according to claim 1, wherein the system comprises:
a first collection module, wherein the first collection module is configured to collect a pupil-wave when a subject is in a calm state and set as a standard pupil-wave;
a first obtaining module, wherein the first obtaining module is configured to measure mental state emotional indicators;
a second obtaining module, wherein the second obtaining module is configured to obtain a plurality of emotions corresponding to the emotional indicators;
a second collection module, wherein the second collection module is configured to collect the pupil-wave of the subject in each emotional state and set as an emotional pupil-wave;
a data processing module, wherein the data processing module is configured to calculate the index values of the emotional indicators based on the standard pupil-wave and the emotional pupil-wave.

8. The system according to claim 7, wherein in the method, the emotional indicators comprise at least one of depression indicators, anxiety indicators and stress indicators:
the depression indicators correspond to a plurality of first emotions, comprising happiness and sadness;
the anxiety indicators correspond to a plurality of second emotions, comprising happiness and threat;
the stress indicators correspond to a plurality of third emotions, comprising happiness and tension.

9. The system according to claim 8, wherein in the method, the steps for collecting pupil-wave comprise:
deriving a raw pupil-wave by capturing temporal changes in the subject's pupil diameter or pupil area;
removing points where a pupil is ≤50 pixels, resulting in the raw pupil-wave with missing values;
employing an adjacent mean difference method to fill in the missing values, and the pupil-wave is obtained: a calculation formula for the adjacent mean difference method is as follows:
P(t)=(P(t−1)+P(t+1))/2 (Formula 1)
P(t) is a value of the pupil-wave at the t moment.

10. The system according to claim 9, wherein in the method, calculation formulas of the bandwidth pupil-wave are as follows:
$$\tilde{P} = \frac{1}{m} {\sum }_{t = 1 ∼ m} {p}_{0} ( t ) \qquad ( Formula 2 )$$
$${EP}_{i} ( t ) = {P}_{i} ( t ) - \tilde{P}$$
P₀(t) is a value of the standard pupil-wave at a t second, m is an acquisition time, \tilde{P} is a mean value, Pᵢ(t) is a value of the pupil-wave corresponding to an i emotion at the t second, EPᵢ(t) is a value of the bandwidth pupil-wave corresponding to the i emotion at the t second.

11. The system according to claim 10, wherein the method comprises a calculation formula for the differential pupil-wave, the calculation formula is as follows:
DPᵢ(t)=Pᵢ(t+1)−Pᵢ(t) (Formula 3)
Pᵢ(t) is a value of the emotional pupil-wave corresponding to the i emotion at the t second, Pᵢ(t+1) is a value of the emotional pupil-wave corresponding to the i emotion at the t+1 second, DPᵢ(t) is a value of the differential pupil-wave corresponding to the i emotion at the t second.

12. The system according to claim 11, wherein in the method, the pre-trained deep convolutional neural network comprises:
a pupil-wave channel, wherein the pupil-wave channel is configured to receive the standard pupil-wave and the emotional pupil-wave and extract first features through a first convolutional neural network;
a bandwidth pupil-wave channel, wherein the bandwidth pupil-wave channel is configured to receive the bandwidth pupil-wave and extract second features through a second convolutional neural network;
a differential pupil-wave channel, wherein the differential pupil-wave channel is configured to receive the differential pupil-wave and extract third features through a third convolutional neural network;
a fully connected layer, wherein the fully connected layer is configured to receive the first, second, and third features extracted from the pupil-wave channel, the bandwidth pupil-wave channel, and the differential pupil-wave channel, and outputs the index values of the emotional indicators.
